SEQUENCE LISTING (25 sequences)

SEQ ID NO: 1 - protein, 6 aa, Artificial Sequence
  Synthetic peptide
  MISC_FEATURE (1)..(1): Xaa denotes Ile, Val or Leu.
  MISC_FEATURE (2)..(2): Xaa denotes any amino acid residue.
  MISC_FEATURE (4)..(5): Xaa denotes any amino acid residue.
  MISC_FEATURE (6)..(6): Xaa denotes Leu or Val.
XXYXXX

SEQ ID NO: 2 - protein, 6 aa, Artificial Sequence
  Synthetic peptide
  MISC_FEATURE (2)..(2): Xaa denotes any amino acid residue.
  MISC_FEATURE (4)..(5): Xaa denotes any amino acid residue.
  MISC_FEATURE (6)..(6): Xaa denotes Val or Ile.
TXYXXX

SEQ ID NO: 3 - protein, 4 aa, Artificial Sequence
  Synthetic peptide
  MISC_FEATURE (2)..(3): Xaa denotes any amino acid residue.
YXXM

SEQ ID NO: 4 - protein, 6 aa, Artificial Sequence
  Synthetic peptide
KIEELE

SEQ ID NO: 5 - protein, 41 aa, Artificial Sequence
  Synthetic peptide
AVSLSKMLKKRSPLTTGVYVKMPPTEPECEKQFQPYFIPIN

SEQ ID NO: 6 - protein, 54 aa, Artificial Sequence
  Synthetic peptide
HLWRRQWRPRRFSALEQGIHPPQAQSKIEELEQEPEPEPEPEPEPEPEPEPEQL

SEQ ID NO: 7 - protein, 97 aa, Artificial Sequence
  Synthetic peptide
CSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 8 - protein, 78 aa, Artificial Sequence
  Synthetic peptide
FKWYSHSKEKIQNLSLISLANLPPSGLANAVAEGIRSEENIYTIEENVYEVEEPNEYYCYVSSRQQPSQPLGCRFAMP

SEQ ID NO: 9 - protein, 82 aa, Artificial Sequence
  Synthetic peptide
LTRKKKALRIHSVEGDLRRKSAGQEEWSPSAPSPPGSCVQAEAAPAGLCGEQRGEDCAELHDYFNVLSYRSLGNCSFFTETG

SEQ ID NO: 10 - protein, 111 aa, Artificial Sequence
  Synthetic peptide
RRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 11 - protein, 167 aa, Artificial Sequence
  Synthetic peptide
QHWRQGKHRTLAQRQADFQRPPGAAEPEPKDGGLQRRSSPAADVQGENFCAAVKNTQPEDGVEMDTRSPHDEDPQAVTYAKVKHSRPRREMASPPSPLSGEFLDTKDRQAEEDRQMDTEAAASEAPQDVTYARLHSFTLRQKATEPPPSQEGASPAEPSVYATLAIH

SEQ ID NO: 12 - protein, 120 aa, Artificial Sequence
  Synthetic peptide
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 13 - protein, 101 aa, Artificial Sequence
  Synthetic peptide
HRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 14 - protein, 74 aa, Artificial Sequence
  Synthetic peptide
HFGKTGRASDQRDLTEHKPSVSNHTQDHSNDPPNKMNEVTYSTLNFEAQQPTQPTSASPSLTATEIIYSEVKKQ

SEQ ID NO: 15 - protein, 63 aa, Artificial Sequence
  Synthetic peptide
GSGSRVTELLYRMKRAETYCPRPLLAIHPTEARHKQKIVAPVKQLLNFDLLKLAGDVESNPGP

SEQ ID NO: 16 - protein, 25 aa, Artificial Sequence
  Synthetic peptide
DGFCILYLLLILLMRSGDVETNPGP

SEQ ID NO: 17 - protein, 20 aa, Artificial Sequence
  Synthetic peptide
LLCFMLLLLLSGDVELNPGP

SEQ ID NO: 18 - protein, 20 aa, Artificial Sequence
  Synthetic peptide
HHFMFLLLLLAGDIELNPGP

SEQ ID NO: 19 - protein, 20 aa, Artificial Sequence
  Synthetic peptide
WFLVLLSFILSGDIEVNPGP

SEQ ID NO: 20 - protein, 20 aa, Artificial Sequence
  Synthetic peptide
KNCAMYMLLLSGDVETNPGP

SEQ ID NO: 21 - protein, 20 aa, Artificial Sequence
  Synthetic peptide
MVISQLMLKLAGDVEENPGP

SEQ ID NO: 22 - protein, 22 aa, Artificial Sequence
  Synthetic peptide
GSGATNFSLLKQAGDVEENPGP

SEQ ID NO: 23 - protein, 21 aa, Artificial Sequence
  Synthetic peptide
GSGEGRGSLLTCGDVEENPGP

SEQ ID NO: 24 - protein, 23 aa, Artificial Sequence
  Synthetic peptide
GSGQCTNYALLKLAGDVESNPGP

SEQ ID NO: 25 - protein, 8 aa, Artificial Sequence
  Synthetic peptide
  MISC_FEATURE (2)..(2): Xaa denotes any amino acid residue.
  MISC_FEATURE (4)..(4): Xaa denotes any amino acid residue.
DXEXNPGP

(19) United States
(12) Patent Application Publication
(10) Pub. No.: US 2020/0157173 A1
(43) Pub. Date: May 21, 2020

(54) CHIMERIC ANTIGEN RECEPTOR, REGULATORY CELLS AND METHODS OF USE

(71) Applicant: TRUSTEES OF DARTMOUTH COLLEGE, Hanover, NH (US)

(72) Inventors: Charles L. Sentman, Grantham, NH (US); Benjamine H. Arellano, Lebanon, NH (US)

(21) Appl. No.: 16/715,077

(22) Filed: Dec. 16, 2019

Related U.S. Application Data
(62) Division of application No. 15/762,737, filed on Mar. 23, 2018.
(60) Provisional application No. 62/233,526, filed on Sep. 28, 2015.

(51) Int. Cl. C07K 14/705; A61K 31/7088; C07K 16/28; C07K 14/725; A61K 48/00; C12N 5/0783; A61K 35/17

(52) U.S. Cl. CPC C07K 14/70521; A61K 31/7088; C07K 16/2803; A61K 2039/5158; A61K 48/005; C12N 5/0636; A61K 35/17; C07K 14/7051

(57) ABSTRACT

A chimeric antigen receptor including a co-inhibitory receptor signaling domain, as well as a nucleic acid construct and immune cells expressing the same are described. Kits and methods of using the immune cells in the treatment or amelioration of chronic inflammation or immune-mediated autoimmunity are also provided.

This application claims the benefit of priority of U.S. Provisional Application No. 62/233,526, filed Sep. 28, 2015, the content of which is incorporated herein by reference in its entirety.

### INTRODUCTION

### Background

There exists a set of devastating diseases that are caused by an over-zealous and unchecked immune response. The targeting of self-antigens under normal physiologic conditions can cause a range of serious ailments including type 1 diabetes, multiple sclerosis, rheumatoid arthritis, systemic lupus erythematosus, and autoimmune encephalomyelitis. A relatively new set of autoimmune diseases, categorized as autoinflammatory diseases, have also been characterized including familial Mediterranean fever (FMF), neonatal onset multisystem inflammatory disease (NOMID), tumor necrosis factor (TNF) receptor-associated periodic syndrome (TRAPS), deficiency of the Interleukin-1 receptor antagonist (DIRA) and Behcet's disease. Additional inflammatory diseases that cause morbidity and mortality in a large number of patients include inflammatory bowel disease (Crohn's disease and ulcerative colitis), chronic granulomatous disease (CGD), and the various forms of vasculitis.

In general, dampening the immune response is the ideal treatment for autoimmune and inflammatory diseases and current therapies revolve around the use of steroids, cytokine antagonists, or NSAIDs. Gene therapies may provide a viable biological alternative to directly and specifically inhibit an overactive immune response.

US 2010/0135974 describes a redirected regulatory T lymphocyte endowed with specificity toward a selected target antigen or ligand by expressing a chimeric receptor polypeptide. This reference indicates that the redirected regulatory T cells at sites of inflammation results in suppression of inflammatory conditions, commonly part of organ-specific autoimmune disease. In addition, this reference suggests that redirected regulatory T cells can be triggered or activated to release suppressive cytokines that will result in suppression of any “bystander” effector T-cells, and by this mechanism, quell an ongoing inflammatory/autoimmune response.

US 2003/0077249 discloses an effector cell transformed with DNA coding for one or more chimeric receptors that contain two or more different cytoplasmic signaling components, e.g., CD28, TNF, interferon receptors, GM-CSF, ZAP-70, LFA-1, CD3 gamma, CD5 or CD2, which are not naturally linked and are chosen to act together cooperatively to produce improved activation of the cell. This reference indicates that the binding domain of the chimeric receptor can bind a surface marker expressed on inflammatory cells or an antigen giving rise to autoimmunity.

US 2014/0219975 describes an engineered T cell that expresses a fusion protein receptor composed of two domains that, when displayed on the surface of the cell, can convert a negative signal into a positive signal to the cell. This reference also describes a switch receptor, which when expressed in a cell converts a positive signal into a negative signal in the cell, wherein the switch receptor contains a first domain that comprises a polypeptide that delivers a positive signal; and a second domain that comprises a polypeptide that delivers a negative signal in the cell.

### SUMMARY OF THE INVENTION

This invention provides a chimeric antigen receptor including at least one signaling domain of a co-inhibitory receptor. In some embodiments, the chimeric antigen receptor includes an antigen targeting domain or recognition domain that binds an antigen or ligand at a site of inflammation or autoimmunity. In other embodiments, the chimeric antigen receptor includes a single chain variable fragment that binds an antigen or ligand at a site of inflammation or autoimmunity. In certain embodiments, the at least one signaling domain of a co-inhibitory receptor is from Cytotoxic T-Lymphocyte Antigen 4 (CTLA-4), Lymphocyte-Activation Gene 3 (LAG-3), Programmed cell death protein 1 (PD-1), T cell Immunoglobulin Mucin-3 (TIM-3), I-cell immunoreceptor with immunoglobulin (TIGIT), B- and T-lymphocyte Attenuator (BILA), leukocyte immunoglobulin-like receptor subfamily B member 4 (LILRB4), LILRB3, CD160, 2B4, Leukocyte-Associated Immunoglobulin-like Receptor 1 (LAIR-1), V-domain Ig suppressor of T cell activation (VISTA), CD66a (CEACAM1), neuropilin-1 (NRp1) or CD44. A nucleic acid construct (e.g., a vector) and an immune cell (e.g., a T lymphocyte) harboring nucleic acids encoding the chimeric antigen receptor are also provided, as are kits and a method for treating chronic inflammation or immune-mediated autoimmunity.

### BRIEF DESCRIPTION OF THE DRAWINGS

FIGS. 1A-1C show that a CAR construct composed of nucleic acids encoding the NKp30 antigen-specific targeting domain fused to the transmembrane and cytoplasmic domains of CTLA-4 and cytoplasmic domain of CD3ζ (NKp30.CTLA4.3Z) is highly expressed on E86 packaging cell lines (FIG. 1C). NKp30 ligand expression on the surface of the cell lines was measured by flow cytometry (FIG. 1A). The isotype control is also shown (FIG. 1B). The percentage of NKp30⁺ cells is indicated.

### DETAILED DESCRIPTION OF THE INVENTION

The present invention is directed to a chimeric antigen receptor and chimeric immune cells that reduce or inhibit inflammation and disease progression. in particular, this invention provides chimeric immune cells that express at least one chimeric antigen receptor (CAR) that binds a specific ligand or antigen expressed at the site of inflammation and/or autoimmunity, wherein said CAR includes at least one co-inhibitory receptor signaling domain. Binding of the CAR to the ligand or antigen localizes the chimeric immune cells to an inflammatory site. By signaling via the activated CAR, the co-inhibitory receptor signaling domain mediates anti-inflammatory effects or inhibits the function and proliferation of a pro-inflammatory immune response within the chimeric immune cell's environment. When using two or more CARs with different signaling domains, CAR engagement can activate multiple signaling pathways. In this respect, the chimeric immune cells of this invention are useful in treatment or amelioration of diseases where chronic inflammation or immune-mediated autoimmunity leads to tissue damage or tissue pathology.

This invention provides nucleic acid constructs and immune cells, which harbor nucleic acids encoding at least one CAR having at least one co-inhibitory receptor signaling domain. A chimeric antigen receptor, also known as an artificial T cell receptor, chimeric T cell receptor, or chimeric immunoreceptor, of this invention is a fusion protein composed of at least one antigen or ligand targeting domain or recognition domain, a transmembrane region that anchors the targeting or recognition domain to the cell surface, at least one signaling domain and an optional extracellular spacer/hinge domain between the targeting domain or recognition domain and the transmembrane region. While some embodiments embrace a nucleic acid construct or immune cell harboring nucleic acids encoding one CAR, other embodiments, include the use of multiple CARs, a CAR with multiple targeting or recognition domains, a CAR with multiple signaling domains, or a CAR with multiple targeting or recognition domains and multiple signaling domains.

Antigen targeting or recognition by CAR molecules of the invention can involve the use of a single chain variable fragment (scFv) that has been assembled from a monoclonal antibody. Alternatively, CARs of the invention can include domains that target ligands (Altenschmidt, et al. (1996) Clin. Cancer Res. 2:1001-8; Muniappan, et al. (2000) Cancer Gene Ther. 7:128-134), peptides (Pameijer, et al. (2007) Cancer Gene Ther. 14:91-97), chimeric ligands (Davies, et al. (2012) Mol. Med. 18:565-576), receptor derivatives (Scholler, et al. (2012) Sci. Translation. Med. 4: Article IDS 132ra53; Zhang, et al. (2012) J. Immunol. 189:2290-9), and single domain antibodies (Sharifzadeh, et al. (2012) Cancer Res. 72:1844-52). When two or more antigen-specific targeting domains target at least two different antigens, the domains may be arranged in tandem and separated by linker sequences.

Antigens or ligands, which can be used as targets at a site of inflammation or autoimmunity, include molecules specific for inflammatory diseases or autoimmune diseases, as well as tissue- or cell-specific molecules. Examples of antigens or ligands specific for inflammatory diseases or autoimmune diseases, which may be targeted by the CARs of the invention include, but are not limited to, one or more of the antigens listed in Table 1.

TABLE 1

| Disease or Condition | Target |
|---|---|
| Inflammatory bowel disease | Antigen or ligand expressed |
| (IBD) | in diseased colon or ileum |
| Rheumatoid arthritis | Antigen or ligand is an |
| | epitope of collagen or an |
| | antigen present in joints, |
| | e.g., Rheumatoid factor IgG |
| | complexes |
| Type I diabetes mellitus or | Pancreatic β cell antigen |
| autoimmune insulitis | and/or insulin |
| Multiple sclerosis | A myelin basic protein (MBP) |
| | antigen or MOG-I or MOG2-2, |
| | proteolipid protein, myelin |
| | oligodendrocyte glycoprotein |
| | and/or a neuronal antigen |
| Autoimmune uveitis or | S-antigen or another uveal or |
| uveoretinitis | retinal antigen |
| Autoimmune orchitis | Testicular antigen |
| Autoimmune oophoritis | An ovarian antigen |
| Psoriasis | Keratinocyte antigen or |
| | another antigen present in |
| | dermis or epidermis |
| Vitiligo | Melanocyte antigen such as |
| | melanin or tyrosinase |
| Autoimmune prostatitis | Prostate antigen |
| Autoimmune hemolytic anemia | Rh blood group antigen |
| Autoimmune thrombocytopenic | Platelet integrin GpIIb:IIIa |
| purpura | |
| Goodpasture's syndrome | Noncollagenous domain of |
| | basement membrane collagen |
| | type IV |
| Pemphigus vulgaris | Epidermal cadherin |
| Graves' Disease | Thyroid Peroxidase and/or |
| | thyroid-stimulating hormone |
| | receptor |
| Hashimoto Thyroiditis | Thyroglobulin |
| Systemic Vasculitides | Myeloperoxidase |
| Crohn's Disease | Glycoprotein 2 |
| Primary Biliary Cirrhosis | M2 or components thereof |
| | (e.g., BCOADC-E2, OGDC-E2, |
| | PDC-E2), Sp100, Gp210 and/or |
| | Nup62 |
| Autoimmunc Hepatitis II | Formiminotransferase |
| | Cyclodeaminase and/or |
| | Cytochrome P450 2D6 |
| Celiac Disease | Tissue transglutaminase |
| | and/or gliadin |
| Thromboembolic Syndrome | β2 Glycoprotein I |
| Systemic Vasculitides/ | Proteinase 3 |
| Wegener's Granulomatosis | |

When targeting an inflammatory response, the target of the CAR of the invention can be an antigen expressed on or by a dendritic cell, macrophage/monocyte, granulocyte or eosinophil present at the inflammation site. Such antigens include, but are not limited to, AOC3 (VAP-1), CCL11 (eotaxin-1), CD20, CD3, CD4, CD5, IFN-γ, IgE, IgE Fc region, IL-1, IL-12, IL-23, IL-17, IL-17A, IL-22, IL-6, integrin α4β7, LFA-1 (CD11a), OX-40L, and TNF-α.

When targeting particular tissues or cells, the target of the CAR of the invention can be a biomarker or cell ligand including, but not limited to, those listed in Table 2.

TABLE 2

| Tissue | Biomarker/Cell Ligand |
|---|---|
| Lung^{a} | FGFR2 |
| | Sprouty Homolog 2 (SPRY2) |
| | Stimulated by retinoic acid 6 (STRA6) |
| | CD68 |
| | Mucin 1 |
| | Bone morphogenetic Protein |
| | Receptor Type 1A |
| Neuron^{a} | S-100 protein |
| Cardiovascular | Caveolin 1 |
| System | |
| Liver^{a} | SLCO1B1 |
| | CYP1A2 |
| | CYP3A4 |
| Kidney^{a} | Glomeruli (NPHS2) |
| Neurons | Myelin Basic Protein |
| (CNS) ^{a} | Myelin Oligodendrocyte Glycoprotein |
| | Proteolipid Protein |
| Glial cells | Aquaporin-4 (AQP4) |
| (CNS) ^{a} | |
| Heart^{a} | β-adrenergic receptor |
| Pancreas/Islet | Retinoic acid early inducible (RAE)-1 |
| Cells^{b} | |
| minor salivary | B7H6 |
| glands^{c} | |
| Liver (bile | Carbonic anhydrase IX (CIAX) |
| duct epithelial | |
| cells) ^{a} | |
| CNS^{a} | Mutant Superoxide dismutase (SOD)-1 |
| Inflammatory | Autoimmune TCRs |
| tissues^{a} | |
| Inflammatory | MHCI/II + Autoimmune peptide |
| tissues^{d} | |

^{a}ScFv as antigen-specific targeting domain;
^{b}NKG2D as antigen-specific targeting domain;
^{c}NKp30 as antigen-specific targeting domain;
^{d}Autoimmune TCR as antigen-specific targeting domain.

Exemplary antigen-specific targeting domains of the instant CAR include, but are not limited to, Cam-3001, CD125, CD154, CD2, CD23 (IgE receptor), CD25 (a chain of IL-2 receptor), IL-6 receptor, rhuMAb β7, OX-40, NKG2D, NKp30, and autoimmune TCR.

In addition to antigen-specific approaches, two “universal” CAR systems have been described. These generic CARs containing avidin (Urbanska, et al. (2012) Cancer Res. 72:1844-52) or antifluorescein isothiocyanate (FITC) scFv (Ang, et al. (2011) Mol. Ther. 19: abstract 353; Chmielewski, et al. (2004) J. Immunol. 173:7647-7653), enabling their use in conjunction with separate targeting moieties that have been biotinylated or conjugated to FITC, respectively.

In embodiments wherein the antigen targeting domain is a scFv, the scFv can be derived from the variable heavy chain (VH) and variable light chain (VL) regions of an antigen-specific mAb linked by a flexible linker. The scFv retains the same specificity as the full antibody from which it was derived (Muniappan, et al. (2000) Cancer Gene Ther. 7:128-134). Various methods for preparing a scFv can be used including methods described in U.S. Pat. No. 4,694,778; Bird, et al. (1988) Science 242:423-442; Ward, et al. (1989) Nature 334:54454; and Skerra, et al. (1988) Science 242:1038-1041. In certain embodiments, the scFv is humanized or is a fully human scFv.

As indicated, the CAR of the invention may also have an extracellular spacer/hinge domain and a transmembrane region or domain. The transmembrane domain may be derived from a natural polypeptide, or may be artificially designed. The transmembrane domain derived from a natural polypeptide can be obtained from any membrane-binding or transmembrane protein. For example, a transmembrane domain of a T cell receptor α or β chain, a CD3ζ chain, CD28, CD3ε, CD45, CD4, CD5, CD7, CD8, CD9, CD16, CD22, CD33, CD37, CD64, CD80, CD86, CD134, CD137, ICOS, CD154, H2-Kb, FcεRIγ or GITR can be used. See, e.g., Kahlon, et al. (2004) Cancer Res. 64:9160-9166; Schambach, et al. (2009) Methods Mol. Biol. 506:191-205; Jensen, et al. (1998) Biol. Blood Marrow Transplant 4:75-83; Patel, et al. (1999) Gene Ther. 6:412; Song, et al. (2012) Blood 119:696-706; Carpenito, et al. (2009) Proc. Natl. Acad. Sci. USA 106:3360-5; Hombach, et al. (2012) Oncoimmunology 1:458-66) and Geiger, et al. (2001) Blood 98:2364-71. The artificially designed transmembrane domain is a polypeptide mainly composed of hydrophobic residues such as leucine and valine. It is preferable that a triplet of phenylalanine, tryptophan and valine is found at each end of the synthetic transmembrane domain. See, U.S. Pat. No. 7,052,906. In one embodiment, the transmembrane domain is composed of residues 153 to 180 of CD28 (GENBANK Accession No. NP_006130). As another embodiment, the transmembrane domain is composed of residues 162 to 183 of a GITR (GENBANK Accession No. NP_004186).

In the CAR of the invention, a spacer or hinge domain can be arranged between the extracellular antigen targeting domain and the transmembrane domain, and/o/between the intracellular signaling domain and the transmembrane domain. A spacer domain refers to any oligopeptide or polypeptide that serves to link the transmembrane domain with the antigen targeting domain and/or the transmembrane domain with the intracellular signaling domain. The spacer domain can be up to 300 amino acids, preferably 10 to 100 amino acids, 25 to 50 amino acids or 2 to 10 amino acids in length.

The spacer domain may have a sequence that promotes binding of a CAR with an antigen or ligand and enhances signaling in a cell. Examples of an amino acid that is expected to promote the binding include cysteine, a charged amino acid, and serine and threonine in a potential glycosylation site, and these amino acids can be used as an amino acid constituting the spacer domain.

As the spacer domain, all or a part of residues 118 to 178 of CD8α (GENBANK Accession No. NP_001759.3), residues 135 to 195 of CD8p (GENBANK Accession No. AAA35664), residues 315 to 396 of CD4 (GENBANK Accession No. NP_000607.1), or residues 137 to 152 of CD28 (GENBANK Accession No. NP_006130.1) can be used. Also, as the spacer domain, a part of a constant region of an antibody H chain or L chain (CH1 region or CL region) can be used. Further, the spacer domain may be an artificially synthesized sequence.

The intracellular signaling domain used in this invention is a molecule that can transmit a signal into a cell when the extracellular antigen or ligand targeting domain present within the same molecule binds to (interacts with) an antigen or ligand. In accordance with the present invention, the CAR includes at least one signaling domain of a co-inhibitory receptor. As is known in the art, a co-inhibitory receptor attenuates and counterbalances activation signals initiated by stimulatory receptors (Thaventhiran, et al. (2012) J. Clin. Cell Immunol. S12:004; Chen & Flies (2013) Nat. Rev. Immunol. 13(4):227-242). The subsequent outcomes on T cell function can range from temporary inhibition to permanent inactivation and cell death (Sinclair (1999) Scand. J. Immunol. 50:10-13). The majority of co-inhibitory receptors belong to the immunoglobulin (Ig) superfamily (Odorizzi & Wherry (2012) J. Immunol. 188:2957-65). There are primarily three mechanisms that are used by membrane bound inhibitory receptors in T cells. The first mechanism involves the sequestration of the ligands for co-stimulatory receptors, depriving the T cell from receiving activation signals necessary for complete activation. The second mechanism involves the recruitment of intracellular phosphatases by one or more of the motifs listed in Table 3 that make up the cytoplasmic tail of certain inhibitory receptors, which dephosphorylate signaling molecules downstream of the T cell receptor and co-stimulatory pathways, leading to a quantitative reduction in activation-induced gene expression. The third mechanism involves the upregulation or down-regulation of genes that code for proteins involved in the inhibition of immune functions including, e.g., basic leucine zipper transcription factor, activating transcription factor-like (BATF) (Odorizzi & Wherry (2012) J. Immunol. 188:2957-65). A co-inhibitory receptor of this invention could use a combination of the above and possibly other yet to be discovered mechanisms to regulate T cell signaling.

TABLE 3

| | | SEQ | Co- |
|---|---|---|---|
| | | ID | stimulatory |
| Motif | Sequence | NO: | receptor |
| Immunoreceptor | (Ile/Val/Leu)-Xaa-Tyr- | 1 | PD-1, BTLA, |
| tyrosine-based | Xaa-Xaa-(Leu/Val) | | LAIR-1, |
| inhibition | | | TIGIT |
| motif (ITIM) | | | |
| Immunoreceptor | Thr-Xaa-Tyr-Xaa-Xaa- | 2 | PD-1, BTLA, |
| tyrosine-based | (Val/Ile) | | 2B4 |
| switch motif | | | |
| (ITSM) | | | |
| YxxM | Tyr-Xaa-Xaa-Met | 3 | CTLA-4 |
| KIEELE | Lys-Ile-Glu-Glu-Leu-Glu | 4 | LAG-3 |
| Conserved | Tyr235, Tyr242 | | TIM-3 |
| Tyrosine | | | |
| Residue | | | |

Co-inhibitory receptor signaling domains of use in this invention include, but are not limited to, Cytotoxic T-Lymphocyte Antigen 4 (CTLA-4), Lymphocyte-Activation Gene (LAG-3), Programmed cell death protein 1 (PD-1), T cell Immunoglobulin Mucin-3 (TIM-3), T-cell immunoreceptor with immunoglobulin (TIGIT), B- and T-lymphocyte Attenuator (BTLA), leukocyte immunoglobulin-like receptor subfamily B member 4 (LILRB4), LILRB3, CD160, 2B4, Leukocyte-Associated Immunoglobulin-like Receptor 1 (LAIR-1), CD66a (CEACAM1), neuropilin-1 (NRp1), CD44, or a combination thereof.

CTLA-4 engagement has been shown to result in a reduction in ZAP70 microcluster formation, calcium mobilization and IL-2 production (Rudd (2008) Nat. Rev. Immunol. 8:153-160). CTLA-4 can also act in a cell-extrinsic manner to regulate T cell responses through altering antigen presenting cell (APC) function. In particular, it has been demonstrated that CD80 and CD86 on APCs can be captured and degraded by CTLA-4 via a process of trans-endocytosis thereby resulting in impaired T cell responses (Qureshi, et al. (2011) Science 332:600-3). Furthermore, CTLA-4 signaling has also been shown to interfere with proximal TCR signaling by suppression of extracellular signal-regulated kinase (ERK) and Jun NH₂-terminal kinase (JNK) activity (Calvo, et al. (1997) J. Exp. Med. 186:1645-53). In addition, CTLA-4 ligation has been shown to attenuate AP-1, NFAT and NF-κB nuclear transcription factor activity in activated CD4⁺ T cells and inhibit DNA-binding of AP-1 and NFAT complexes in the nucleus (Fraser, et al. (1999) Eur. J. Immunol. 29:838-44). Moreover, the CTLA-4 has been shown to regulate T cell tolerance or autoimmunity. In particular, CTLA-4 has been shown to regulate relapsing-remitting experimental autoimmune encephalomyelitis (Karandikar, et al. (1996) J. Exp. Med. 184:783-8); CTLA-4 blockade results in enhanced production of the encephalitogenic cytokines TNF-α, IFN-γ and IL-2 (Perrin, et al. (1996) J. Immunol. 157:1333-6). CTLA-4 has also been reported to play an important role in controlling the progression of autoimmunity in the BDC2.5 non-obese diabetic (NOD) TCR transgenic model of diabetes (Luhder, et al. (1998) J. Exp. Med. 187:427-32). In this model, treatment with anti-CTLA-4 monoclonal antibody induced diabetes rapidly, but only if treatment occurred before the onset of insulitis (Luhder, et al. (1998) J. Exp. Med. 187:427-32). Of particular relevance to the present invention, it has been shown that CTLA-4 engagement directly enhances Foxp3 induction in CD4⁺ T cells. More specifically, naïve T cells activated with plate bound α-CD3, TGF-β, and IL-2 express more Foxp3 when α-CTLA-4 is present during stimulation (Barnes, et al. (2013) Mucosal Immunol. 6(2):324-34). Therefore, when expressed in an immune cell, a CAR containing the co-inhibitory signaling domain of CTLA-4 can activate the immune cell toward an immune suppressive phenotype. An exemplary CTLA-4 signaling domain of use in this invention has the following amino acid sequence: AVSLSKML KKRSPLTTGV YVKMPPTEPE CEKQFQPYFI PIN (SEQ ID NO:5). See also, GENBANK Accession Nos. NP_005205 and NP_001032720.

LAG-3 expressed on the cell surface can be cleaved within the transmembrane domain at the connecting peptide (CP) by two members of the TNF converting enzyme (TACE) family of metalloproteases known as ADAM10 and ADAM17 (Li, et al. (2007) EMBO J. 26:494-504), to release soluble LAG-3 (sLAG-3). sLAG-3 has been shown to reduce the differentiation of monocytes into macrophages in the presence of granulocyte-macrophage colony-stimulating factor (GM-CSF) as well as their differentiation into dendritic cells in the presence of GM-CSF and interleukin-4 (Buisson & Triebel (2005) Immunology 114:369-374). Dendritic cells derived from monocytes in the presence of sLAG-3 show impaired antigen-presentation function, as assessed by the reduced capability to induce proliferation of T cells. Further, similar to the protein kinase C binding site in the CD4 molecule, the cytoplasmic tail of LAG-3 contains a region with a potential serine phosphorylation site. Another motif that has been identified in the intracytoplasmic region of LAG-3 is an unusual ‘EP’ (glutamic acid-proline) repeat that binds a protein termed LAP (LAG-3-associated protein) and is predicted to be important in the anchorage of the immune synapse to the microtubule network following TCR engagement (Triebel. (2003) Trends Immunol. 24:619-22). These properties indicate that the LAG-3 cytoplasmic domain mediates intracellular signal transduction and/or molecular aggregation (Workman, et al. (2002) J. Immunol. 169:5392-5). An exemplary LAG-3 signaling domain of use in this invention has the following amino acid sequence: HLWRRQWRP RRFSALEQGI HPPQAQSKIE ELEQEPEPEP EPEPEPEPEP EPEQL (SEQ ID NO:6). See also, GENBANK Accession No. NP_002277.

PD-1 signaling inhibits alloreactive T cell activation, can promote induced regulatory T cell development and enhance Treg function in lymphoid organs and tissues that are targets of autoimmune attack (Riella, et al. (2012) Am. J. Tranplant. 12:2575-87; Francisco, et al. (2010) Immunol. Rev. 236:219-242). Upon PD-1 ligation, both of the cytoplasmic ITIM and ITSM tyrosine motifs are phosphorylated possibly by Lck and/or C-terminal Src kinase with a consequent recruitment of SHP-2. This reduces the TCR-triggered phosphorylation of CD3ζ, ZAP70 and PKCθ, while also blocking the CD28-mediated activation of Phosphoinositide 3-kinase (PI3K) and Akt (Sheppard, et al. (2004) FEBS Lett. 574:37-41). The inhibitory signal mediated by PD-1 depends on the strength of the TCR signal with much stronger phosphorylation of PD-1 and association with SHP-2 observed at high levels of TCR stimulation. PD-1 ligation interferes with the induction of the cell survival factor Bcl-xL (Chemnitz, et al. (2004) J. Immunol. 173:945-54) and the expression transcription factors associated with effector cell function such as GATA-3, T-bet and Eomes (Keir, et al. (2008) Annu. Rev. Immunol. 26:677-704). PD-1 ligation can also block cell cycle progression and proliferation of T cells by interfering with multiple regulators of the cell cycle via a mechanism that uses the suppression of transcription factors in order to down-regulate genes that code for proteins involved in cell cycle control. Moreover, PD-1-mediated co-inhibitory signals have been described as being involved in Foxp3 induction (Wang, et al. (2008) Proc. Natl. Acad. Sci. USA 105:9331-6). An exemplary PD-1 signaling domain of use in this invention has the following amino acid sequence: CSRAARGTI GARRTGQPLK EDPSAVPVFS VDYGELDFQW REKTPEPPVP CVPEQTEYAT IVFPSGMGTS SPARRGSADG PRSAQPLRPE DGHCSWPL (SEQ ID NO:7). See also, GENBANK Accession No. NP_005009.

TIM-3 has been shown to negatively regulate macrophage activation, and TIM-3 signaling on cells of the innate immune system critically influences regulation of adaptive immune responses (Frisancho-Kiss, et al. (2009) Brain Behav. Immun. 23:649-657; Frisancho-Kiss, et al. (2006) J. Immunol. 176:6411-6415; Monney, et al. (2002) Nature 415:536-541; Seki, et al. (2008) Clin. Immunol. 127:78-88). For example, in vivo administration of TIM-3 antibody enhances a Th1-dependent autoimmune encephalomyelitis by increasing the number and activation of macrophages (Monney, et al. (2002) Nature 415:536-541). Blocking TIM-3 signaling during an innate immune response to viral infection reduces CD80 costimulatory molecule expression on macrophages, leading to a decreased CTLA-4 level in CD4⁺ T cells, decreased Tregs, and increased inflammatory heart disease (Frisancho-Kiss, et al. (2006) J. Immunol. 176:6411-6415). Further, galectin-9 triggering of TIM-3 has been shown to attenuate Th1 and Th17 responses in disease models of skin inflammation (Niwa, et al. (2009) Clin. Immunol. 132:184-94), experimental autoimmune arthritis (Seki, et al. (2008) Clin. Immunol. 127:78-88) and herpes simplex virus-induced ocular inflammation (Sehrawat, et al. (2009) J. Immunol. 182:3191-201). An exemplary TIM-3 signaling domain of use in this invention has the following amino acid sequence: FKWYSHS KEKIQNLSLI SLANLPPSGL ANAVAEGIRS EENIYTIEEN VYEVEEPNEY YCYVSSRQQP SQPLGCRFAMP (SEQ ID NO:8). See also, GENBANK Accession No. NP_116171.

TIGIT has been indicated to mediate T cell-intrinsic inhibitory effects, predominantly through the activation and phosphorylation of ERK (Stengel, et al. (2012) Proc. Natl. Acad. Sci. USA 109:5399-5404). TIGIT deficient mice has been shown to aggravate experimental autoimmune encephalomyelitis (EAE) through hyperproliferative T cell responses, proinflammatory cytokine production (e.g., IL-6, IFN-γ and IL-17), and increased susceptibility to autoimmunity (Joller, et al. (2011) J. Immunol. 186:1338-1342). By comparison, stimulation of the TIGIT receptor on T cells with agonistic monoclonal antibodies demonstrated a direct inhibitory effect on cell cycle entry, a decrease in the expression of transcription factors such as T-bet, GATA3, IFN regulatory factor 4 (IRF4) and retinoic acid-related orphan receptor c (RORc), with a consequent inhibition of proinflammatory (IFN-γ) cytokine production (Lozano, et al. (2012) J. Immunol. 188:3869-75). In line with this finding, it has been shown that TIGIT overexpression reduces the development of EAE (Levin, et al. (2011) Eur. J. Immunol. 41:902-915). Furthermore, soluble TIGIT inhibits collagen-induced arthritis by dampening CD4⁺ T cell responses and by interfering with CD226-mediated co-stimulation. Moreover, exposure of regulatory T cells to an agonistic anti-TIGIT antibody triggers a 2-fold increase in IL-10 and Fgl2 gene expression by the regulatory T cells in vitro (Joller, et al. (2014) Immunity 40:569-81). An exemplary TIGIT signaling domain of use in this invention has the following amino acid sequence: LTRKKKAL RIHSVEGDLR RKSAGQEEWS PSAPSPPGSC VQAEAAPAGL CGEQRGEDCA ELHDYFNVLS YRSLGNCSFF TETG (SEQ ID NO:9). See also, GENBANK Accession No. NP_776160.

BTLA contains an ITIM, an ITSM and two Grb2-binding motifs in its cytoplasmic domain (Chemnitz, et al. (2006) J. Immunol. 176:6603-14; Riley (2009) Immunol. Rev. 229:114-25; Riley & June (2005) Blood 105:13-21). BTLA is capable of recruiting phosphatase to dampen T cell signaling (Sedy, et al. (2005) Nat. Immunol. 6:90-98). In vitro, Hvem^{-/-} and Btla^{-/-} T cells are hyper-responsive to TCR stimulation. In vivo, Hvem^{-/-} and Btla^{-/-} mice show increased susceptibility to the induction of EAE by injection of myelin oligodendrocyte glyco-protein (Wang, et al. (2005) J. Clin. Invest. 115:711-717; Watanabe, et al. (2003) Nat. Immunol. 4:670-679). In addition, BTLA signaling has been shown to limit T cell activity in vivo and negatively regulates homeostatic expansion of CD4⁺ and CD8⁺ T cells (Krieg, et al. (2007) Nat. Immunol. 8:162-171). An exemplary BTLA signaling domain of use in this invention has the following amino acid sequence: RR HQGKQNELSD TAGREINLVD AHLKSEQTEA STRQNSQVLL SETGIYDNDP DLCFRMQEGS EVYSNPCLEE NKPGIVYASL NHSVIGPNSR LARNVKEAPT EYASICVRS (SEQ ID NO:10). See also, GENBANK Accession Nos. NP_861445 and NP_001078826.

LILRB4 (also known as gp49B) inhibits IgE-dependent activation of mast cells in vitro through its two ITIMs that recruit the src homology domain type-2-containing tyrosine phosphatase 1 into the cell membrane. Lilrb4^{-/-} mice have been shown to exhibit greater incidence and severity of IgE⁻ and mast cell-dependent anaphylactic inflammation compared with mice that express LTLRB4. In addition, mast cell-dependent inflammation induced by the interaction of stem cell factor (SCF) with its receptor Kit is also more severe in Lilrb4^{-/-} mice, indicating that the counter-regulatory function of LILRB4 extends beyond inflammation induced by Fc receptors, which signal through ITIMs, to responses initiated through a receptor tyrosine kinase (Katz (2007) Immunol. Rev. 217:222-30). Furthermore, Lilrb4^{-/-} mice exhibit increased susceptibility to collagen-induced arthritis and LPS-induced septic shock (Zhou, et al. (2005) Eur. J. Immunol. 35:1530-1538; Katz (2007) Immunol. Rev. 217:222-30). An exemplary LILRB4 signaling domain of use in this invention has the following amino acid sequence: QHWRQGKHRT LAQRQADFQR PPGAAEPEPK DGGLQRRSSP AADVQGENFC AAVKNTQPED GVEMDTRSPH DEDPQAVTYA KVKHSRPRRE MASPPSPLSG EFLDTKDRQA EEDRQMDTEA AASEAPQDVT YARLHSFTLR QKATEPPPSQ EGASPAEPSV YATLAIH (SEQ ID NO:11). See also, GENBANK Accession Nos. NP_001265355, NP_001265356, NP_001265357, NP_001265358 and NP_001265359.

LILRB3, also known as PIR-B, possesses ITIMs within its cytoplasmic domain. These domains bind and activate intracellular phosphatases including Src-homology 2 (SH2)-domain-containing tyrosine phosphatase 1 (SHP-1) and SHP-2 inhibit activating-type receptor-mediated signaling (Blery, et al. (1998) Proc. Natl. Acad. Sci. USA 95:2446-51). Further, pirb gene deletion causes exaggerated dextran sodium sulfate (DSS)-induced colonic injury, which is dependent on the expression of PIR-B on macrophages (Munitz, et al. (2010) Gastroenterology 139:530-41). PIR-B also plays a role in DC maturation because knockout mice lacking PIR-B display perturbations in DC development and altered Th1 and Th2 immune responses (Ujike, et al. (2002) Nat. Immunol. 3:542). Further, PIR-B served as a permissive checkpoint for IL-5-induced development of eosinophils by suppressing the proapoptotic activities of PIR-A, which are mediated by the Grb2-Erk-Bim pathway. In particular, Pirb^{-/-} mice display impaired aeroallergen-induced lung eosinophilia and induction of lung T_{H}2 cell responses, thereby demonstrating a role for PIR-B in eosinophil-associated diseases (Baruch-Morgenstern, et al. (2014) Nat. Immunol. 15:36-44). Exemplary LILRB3 signaling domains of use in this invention are provided under GENBANK Accession Nos. NP_001074919 and NP_006855.

CD160, a glycosylphosphatidylinositol-linked receptor, binds HVEM and inhibits CD4+ proliferation and cytokine production (Cai, et al. (2008) Nat. Immunol. 9:176-185). CD160 is highly expressed on human NK cell subsets, CD8+ T cells, NKT cells, γδ T cells, and on all intestinal intra-epithelial T lymphocytes (IELs; Maeda, et al. (2005) J. Immunol. 175: 4426-4432). Further, CD8 T-cell populations expressing CD160 have reduced proliferation capacity and perforin expression (Vigano, et al. (2014) PLoS Pathog. 10(9):e1004380). An exemplary CD160 signaling domain of use in this invention is provided under GENBANK Accession No. NP_008984.

2B4 (also known as CD244 or SLAMf4) is a 38-kD type I transmembrane protein and member of the CD2 subset of the immunoglobulin superfamily molecules (Lee, et al. (2004) J. Exp. Med. 199:1245-1254; Vaidya, et al. (2005) J. Immunol. 174:800-807). 2B4 is expressed on NK cells, monocytes, basophils, and eosinophils, and is inducibly expressed on a subset of CD8⁺ T cells in both mice and humans (Rey, et al. (2006) Eur. J. Immunol. 36:2359-2366; Wherry, et al. (2007) Immunity 27:670-684; Blackburn, et al. (2009) Nat. Immunol. 10:29-37; Bengsch, et al. (2010) PLoS Pathog. 6:e1000947; Raziorrouh, et al. (2010) Hepatology 52:1934-1947; Waggoner, et al. (2010) J. Clin. Invest. 120:1925-1938; Wang, et al. (2010) J. Immunol. 185:5683-5687). In NK cells, 2B4 has been reported to have both activating and inhibitory functions (Laouar, et al. (2007) J. Immunol. 178:652-656); however evidence in both murine and human models indicates that its role in T cells is co-inhibitory. 2B4 expression is reduced in patients with systemic lupus erythematosus (SLE; Kim, et al. (2010) Clin. Exp. Immunol. 160:348-358), and 2B4 deficiency in mice results in spontaneous development of a SLE-like disease in autoimmune-prone genetic backgrounds (Brown, et al. (2011) J. Immunol. 187:21-25). An exemplary 2B4 signaling domain of use in this invention has the following amino acid sequence: WRRKR KEKQSETSPK EFLTIYEDVK DLKTRRNHEQ EQTFPGGGST IYSMIQSQSS APTSQEPAYT LYSLIQPSRK SGSRKRNHSP SFNSTIYEVI GKSQPKAQNP ARLSRKELEN FDVYS (SEQ ID NO:12). See also, GENBANK Accession Nos. NP_057466, NP_001160135 and NP_001160136.

LAIR-1 (also known as CD305) can inhibit TCR-mediated signals through the recruitment of C-terminal Csk, one or more of the phosphatases SHIP, SHP-1 or SHP-2, and to a certain extent on signaling through p38 MAP kinase and ERK signaling (Maasho, et al. (2005) Mol. Immunol. 42:1521-30). LAIR-1 has been shown to exist in a basally tyrosine phosphorylated state that constitutively recruits and activates SHP-1, which can then dephosphorylate downstream TCR signaling components and influence the basal threshold of T cell activation (Jansen, et al. (2007) Eur. J. Immunol. 37:914-24; Sathish, et al. (2001) J. Immunol. 166:1763-70). LAIR-1 signaling inhibits IL-4 and IL-10 productions from activated T cells, as well as IFN-γ and IL-2. Further, LAIR-1 signal also decreases the production of IL-10 and transforming growth factor-β (TGF-β) from DCs thereby indicating that LAIR-1 inhibitory effects on allergic responses are mediated by a direct suppression of DC and T-cell functions (Omiya, et al. (2009) Immunology 128:543-555). LAIR-1 thus provides a mechanism to prevent the initiation of immune responses and also for the down-regulation of ongoing immune responses (Maasho, et al. (2005) Mol. Immunol. 42:1521-30). An exemplary LAIR-1 signaling domain of use in this invention has the following amino acid sequence: HRQN QIKQGPPRSK DEEQKPQQRP DLAVDVLERT ADKATVNGLP EKDRETDTSA LAAGSSQEVT YAQLDHWALT QRTARAVSPQ STKPMAESIT YAAVARH (SEQ ID NO:13). See also, GENBANK Accession Nos. NP_002278, NP_068352, NP_001275952, NP_001275954, NP_001275955 and NP_001275956.

CD66a or. CEACAM1 (biliary glycoprotein) is a member of the Ig superfamily and the CEA family of molecules. It is a type I membrane glycoprotein known to mediate homotypic cell adhesion through binding of the most distal IgV-like ectodomain, the N-domain. CEACAM1 is expressed as a number of different splice variants in humans, with one, three, or four extracellular Ig-like domains and a L or S cytoplasmic tail (e.g., CEACAM1-4L is the four domain-long cytoplasmic isoform). The CEACAM1-L encodes two ITIMs, which when phosphorylated, bind SHP-1, and endows CEACAM1 with inhibitory functions in epithelial cells, T cells, B cells, and NK cells (Chen, et al. (2001) J. Leukoc. Biol. 70:335-340; Gray-Owen, et al. (2006) Nat. Rev. Immunol. 6:433-446; Nagaishi, et al. (2006) Immunity 25:769-781; Markel, et al. (2002) J. Immunol. 168:2803-2810; Beauchemin, et al. (1997) Oncogene 14:783-790; Chen & Shively (2004) J. Immunol. 172:3544-3552; Pantelic, et al. (2005) Infect. Immun. 73:4171-4179; Lobo, et al. (2009) J. Leukocyte Biol. 86:205-218). For example, during the time course of human T cell activation, CEACAM1 is induced and corresponds with a decrease in IL-2R expression and the response of T cell lines to IL-2 (Chen & Shively (2004) J. Immunol. 172:3544-3552). Furthermore, enforced expression of CEACAM1 in human Jurkat cells leads to inhibition of proliferation and their activation response to IL-2 (Chen & Shively (2004) J. Immunol. 172:3544-3552; Chen, et al. (2004) J. Immunol. 172:3535-3543). An exemplary CD66a signaling domain of use in this invention has the following amino acid sequence: HFGKTGRA SDQRDLTEHK PSVSNHTQDH SNDPPNKMNE VTYSTLNFEA QQPTQPTSAS PSLTATFIIY SEVKKQ (SEQ ID NO:14). See also, GENBANK Accession Nos. NP_001703, NP_001020083, NP_001171744, NP_001171742, NP_001171745, and NP_001192273.

The cell adhesion molecule CD44, which is the major hyaluronan receptor, has been implicated in the binding, endocytosis, and metabolism of hyaluronan (HA). In bleomycin-induced acute lung injury, CD44-deficient mice show an enhanced and persistent inflammatory response due to impaired clearance of apoptotic neutrophils and HA fragments from the injury site (Teder, et al. (2002) Science 296:155-8). In addition, it has been shown that CD44 negatively regulates in vivo inflammation mediated by Toll-Like Receptors (TLRs) via NF-κB activation, which leads to proinflammatory cytokine production. Furthermore, it has been shown that CD44 directly associates with TLR2 when stimulated by the TLR2 ligand zymosan and that the cytoplasmic domain of CD44 is crucial for its regulatory effect on TLR signaling (Kawana, et al. (2008) J. Immunol. 180:4235-45). Exemplary CD44 signaling domains of use in this invention are provided under GENBANK Accession Nos. NP_000601, NP_001001389, NP_001001390, NP_001001391, NP_001001392, NP_001189484, NP_001189485 and NP_001189486.

Although not characterized as a co-inhibitory receptor, Nrp1 also finds use in the present invention as it has been shown to suppress autoreactive CD4⁺ T cells in a murine experimental autoimmune encephalomyelitis model (Solomon, et al. (2011) Proc. Natl. Acad. Sci. USA 108:2040-2045). In addition, gene-expression analysis reveals an Nrp1-induced transcriptional profile that is consistent with promoting T regulatory cell survival, stability and quiescence, and is similar to a Foxo-dependent transcriptional signature (Delgoffe, et al. (2013) Nature 501:252-6). Exemplary Nrp1 signaling domains of use in this invention are provided under GENBANK Accession Nos. NP_003864, NP_001019799, NP_001019800, NP_001231901, and NP_001231902.

As described above, co-inhibitory receptors have inhibitory effects on proinflammatory immune cells when endogenously expressed. However, in accordance with this invention, the present CAR is exogenously expressed in an immune cell, e.g., a CD4+ T cell, and provides that cell with co-inhibitory signals that activate/drive regulatory immune pathways. In this respect, the CAR aids in activating the T cell toward an immune suppressive phenotype, especially when the CAR is transduced into a Foxp3 expressing T cell.

In a CAR containing more than one intracellular signaling domain, an oligopeptide linker or a polypeptide linker can be inserted between the intracellular signaling domains to link the domains. Preferably, a linker having a length of 2 to 10 amino acids can be used. Particularly, a linker having a glycine-serine continuous sequence can be used.

It is also to be understood that by any particular gene/protein, the invention encompasses the gene/protein and any obvious variants thereby, which may be allelic variants or other modifications, which maintain the immunosuppressive and/or anti-inflammatory activities. The present invention also includes families of the gene/protein, which are related by sequence similarity or function.

In addition to the antigen targeting domain, extracellular spacer/hinge domain, transmembrane domain, and signaling endodomain, the CAR of the invention can also include a signal peptide sequence linked to the N-terminus of the CAR. Signal peptide sequences exist at the N-terminus of many secretory proteins and membrane proteins, and typically have a length of 15 to 30 amino acids. Since many of the protein molecules mentioned above have signal peptide sequences, these signal peptides can be used as a signal peptide for the CAR of this invention.

As will be appreciated by one of skill in the art, in some instances, a few amino acids at the ends of the antigen or ligand targeting domain can be deleted, usually not more than 10, more usually not more than 5 residues. Also, it may be desirable to introduce a small number of amino acids at the borders, usually not more than 10, more usually not more than 5 residues. The deletion or insertion of amino acids will usually be as a result of the needs of the construction, providing for convenient restriction sites, ease of manipulation, improvement in levels of expression, or the like. In addition, the substitute of one or more amino acids with a different amino acid can occur for similar reasons, usually not substituting more than about five amino acids in any one domain.

For the purposes of this invention, “nucleic acids” refer to single or double stranded nucleic acid molecules, which are isolated and provided in the form of RNA, a complementary polynucleotide (cDNA), a genomic polynucleotide and/or a composite polynucleotide (e.g., a combination of the above). As used herein, the term “nucleic acid construct” refers to a nucleic acid molecule, which includes nucleic acids encoding a CAR protein. In some embodiments, the nucleic acid construct is a linear naked molecule or a vector, e.g., a plasmid, a bacmid, a phagemid, a cosmid, a phage, a virus or an artificial chromosome.

In accordance with the present invention, the nucleic acid construct is transformed or introduced into an immune cell and is transcribed and translated to produce a product (e.g., at least one chimeric receptor, and optionally a suicide protein). Thus, the nucleic acid construct further includes at least one promoter for directing transcription of the one or more CARs. According to some embodiments, nucleic acids encoding the at least one CAR are operably linked to a promoter sequence. A coding nucleic acid is “operably linked” to a regulatory sequence (e.g., promoter) if the regulatory sequence is capable of exerting a regulatory effect on the coding sequence linked thereto. In other words, the promoter(s) of the invention is positioned so as to promote transcription of the messenger RNA from the DNA encoding the CAR and anti-inflammatory or immunosuppressant protein.

The promoter(s) of the invention can be of genomic origin or synthetically generated. A variety of promoters for use in T cells have been described in the art. For example, the CD4 promoter is disclosed by Marodon, et al. ((2003) Blood 101(9):3416-23). The promoter can be constitutive or inducible, where induction is associated with the specific cell type, a specific level of maturation, or drug (e.g., tetracycline or doxorubicin). Alternatively, a number of viral promoters are also suitable. Promoters of interest include the β-actin promoter, SV40 early and late promoters, immunoglobulin promoter, human cytomegalovirus promoter, retrovirus promoter, and the Friend spleen focus-forming virus promoter. The promoters may or may not be associated with enhancers, wherein the enhancers may be naturally associated with the particular promoter or associated with a different promoter, e.g., viral LTR, EF1-alpha promoter, or a doxycycline-responsive promoter.

In the nucleic acid construct of the invention, at least one promoter directs transcription of one or more CARs. According to some embodiments, nucleic acids encoding two or more CARs are independently expressed via different promoters, i.e., nucleic acids encoding one CAR are operably linked to a first promoter and nucleic acids encoding a second CAR are operably linked to a second promoter, which may be the same or different than the first promoter. While it is contemplated that two or more CARs may be expressed via different promoters using two or more independent nucleic acid constructs, in accordance with the present invention, it is preferable that the nucleic acids encoding the CARs reside on a single nucleic acid construct. Further, in other embodiments of the invention, nucleic acids encoding two or more CARs are co-expressed via a single promoter, i.e., nucleic acids encoding the two or more CARs are in tandem and operably linked to a single promoter.

The simultaneous or co-expression of two or more CARs via a single promoter may be achieved by the use of an internal ribosomal entry site (IRES) or cis-acting hydrolase element. The term “internal ribosome entry site” or “IRES” defines a sequence motif that promotes attachment of ribosomes to that motif on internal mRNA sequences. Consequently, an mRNA containing an IRES sequence motif results in two translational products, one initiating from the 5′-end of the mRNA and the other by an internal translation mechanism mediated by the IRES. A number of IRES have been described and can be used in the nucleic acid construct of this invention. See, e.g., U.S. Pat. No. 8,192,984; WO 2010/119257; and US 2005/0112095.

A “cis-acting hydrolase element” or “CHYSEL” refers to a peptide sequence that causes a ribosome to release the growing polypeptide chain that it is being synthesizes without dissociation from the mRNA. In this respect, the ribosome continues translating and therefore produces a second polypeptide. Peptides such as the foot and mouth disease virus (FMDV) 2A sequence (GSGSRVTELLYRMKRAETYC PRPLLAIHPTEARHKQKIVAPVKQLLNFDLLKLAGDVESNPGP, SEQ ID NO:15), sea urchin (Strongylocentrotus purpuratus) 2A sequence (DGFCILYLLLILLMRSGDVETNPGP, SEQ ID NO:16); Sponge (Amphimedon queenslandica) 2A sequence (LLCFMLLLLLSGDVELNPGP, SEQ ID NO:17; or HHFMFLLLLL AGDIELNPGP, SEQ ID NO:18); acorn worm (Saccoglossus kowalevskii) (WFLVLLSFILSGDIEVNPGP, SEQ ID NO:19) 2A sequence; amphioxus (Branchiostoma floridae) (KNCAMYMLLLSGDVETNPGP, SEQ ID NO:20; or MVISQLMLKLAGDVEENPGP, SEQ ID NO:21) 2A sequence porcine teschovirus-1 (GSGATNFSLLKQAGDVEENPGP, SEQ ID NO:22) 2A sequence; Thoseaasigna virus (GSGEGRGSLL TCGDVEENPGP, SEQ ID NO:23) 2A sequence; and equine rhinitis A virus (GSGQCTNYALLKLAGDVESNPGP, SEQ ID NO:24) 2A sequence are CHYSELs of use in this invention. In some embodiments, the 2A sequence is a naturally occurring or synthetic sequence that includes the 2A consensus sequence D-X-E-X-NPCP (SEQ ID NO:25), in which X is any amino acid residue. In preferred embodiments, a furin sequence is included upstream of the 2A sequence to allow the proteins to be separated.

The sequence of the open reading frames encoding the CAR and anti-inflammatory or immunosuppressant protein can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA or provide T cell-specific expression (Barthel and Goldfeld (2003) J. Immunol. 171(7):3612-9). Also, it may be further advantageous to use endogenous or exogenous non-coding regions to stabilize the mRNA.

For expression of a CAR, the naturally occurring or endogenous transcriptional initiation region of the nucleic acid sequence encoding N-terminal component of the CAR can be used to generate the CAR in the target host. Alternatively, an exogenous transcriptional initiation region can be used which allows for constitutive or inducible expression, wherein expression can be controlled depending upon the target host, the level of expression desired, the nature of the target host, and the like.

The termination region(s) of the construct may be provided by the naturally occurring or endogenous transcriptional termination regions of the nucleic acids encoding the C-terminal component of the last gene. Alternatively, the termination region may be derived from a different source. For the most part, the source of the termination region is generally not considered to be critical to the expression of a recombinant protein and a wide variety of termination regions can be employed without adversely affecting expression.

In some embodiments of the invention, a nucleic acid construct or cell harboring the nucleic acid construct includes a nucleic acid encoding a protein that is capable of triggering cell death or elimination. Examples of such proteins include suicide proteins such as thymidine kinase (TK) of the HSV virus (herpesvirus) type I (Bonini, et al. (1997) Science 276:1719-1724), a Fas-based “artificial suicide gene” (Thomis, et al. (2001) Blood 97:1249-1257), E. coli cytosine deaminase gene or caspase-9, which are activated by gancyclovir, AP1903, 5-fluorocytosine or a specific chemical inducer of dimerization (CID), respectively.

The nucleic acid encoding the protein for cell death or elimination is advantageously provided in the nucleic acid construct of the invention to allow for the opportunity to ablate the transduced immune cells in case of toxicity and to destroy the chimeric construct once the signs or symptoms of disease have been reduced or ameliorated. The use of suicide genes for eliminating transformed or transduced cells is described in the art. For example, Bonini, et al. ((1997) Science 276:1719-1724) teach that donor lymphocytes transduced with the HSV-TK suicide gene provide antitumor activity in patients for up to one year and elimination of the transduced cells is achieved using ganciclovir. Further, Gonzalez, et al. ((2004) J. Gene Med. 6:704-711) describe the targeting of neuroblastoma with cytotoxic T lymphocyte clones genetically modified to express a chimeric scFvFc: ζ immunoreceptor specific for an epitope on L1-CAM, wherein the construct further expresses the hygromycin thymidine kinase (HyTK) suicide gene to eliminate the transgenic clones.

It is contemplated that the nucleic acid encoding the protein for cell death or elimination can be expressed from the same promoter as the CAR or from a different promoter. Generally, however, nucleic acid encoding the protein for cell death or elimination and CAR reside on the same construct or vector. Expression of the protein for cell death or elimination from the same promoter as the CAR can be accomplished using the IRES or CHYSEL sequences described herein.

In certain embodiments of the invention, a nucleic acid construct or cell harboring the nucleic acid construct uses a detectable marker so that the cell that harbors the nucleic acid construct is identifiable, for example for qualitative and/or quantitative purposes. The detectable marker may be detectable by any suitable means in the art, including by flow cytometry, fluorescence, spectrophotometry, and so forth. An example of a detectable marker is one that encodes a nonfunctional gene product but that is still detectable by flow cytometry means, for example, or can be used to select transgenic cells by flow cytometry or magnetic selection. In addition to detection, the marker protein can be used as a means to eliminate the transduced cells in vivo via an antibody that recognizes the marker protein. Examples of marker proteins of use in cell elimination include, e.g., truncated CD19 (Tey, et al. (2007) Biol. Blood Marrow Transplant 13:913-24), the extracellular region of CD20 (Introna, et al. (2000) Hum. Gene Ther. 11:611-20; Griffioen, et al. (2009) Haematologica 94:1316-20), and the extracellular region of EGFR (Terakura, et al. (2012) Blood 119:72-82). See also, Lang, et al. (2004) Blood 103:3982-5. Incorporation of these proteins into gene-modified T cells renders the cells susceptible to elimination by clinically used anti-CD19 antibodies, anti-CD20 antibodies, and anti-EGFR antibodies (e.g., cetuximab), respectively.

A nucleic acid construct according to the present invention can be produced by any means known in the art, though preferably it is produced using recombinant DNA techniques. Nucleic acids encoding the CAR can be prepared and assembled into a complete coding sequence by standard techniques of molecular cloning (genomic library screening, PCR, primer-assisted ligation, site-directed mutagenesis, etc.). Nucleic acids encoding the other moieities (e.g., IRES or CHYSEL) may be similarly prepared. The resulting nucleic acids are preferably inserted into an expression vector and used to transform suitable mammalian host cells, preferably immune cells such as T lymphocyte cells.

The constructs and immune cells of this invention find application in subjects having or suspected of having an inflammatory condition, in particular a chronic inflammatory condition, or immune-mediated autoimmunity. Chronic inflammatory conditions and autoimmune diseases that can be treated using the immune cells and nucleic acid constructs of this invention include, for example, rheumatoid arthritis, reactive arthritis, multiple sclerosis, Type I diabetes mellitus or autoimmune insulitis, systemic lupus erythematosus, autoimmune uveoretinitis, autoimmune vasculitis, bullous pemphigus, myasthenia gravis, autoimmune thyroiditis or Hashimoto's disease, Sjogren's syndrome, granulomatous orchitis, autoimmune oophoritis, Inflammatory bowel disease, Crohn's disease, sarcoidosis, rheumatic carditis, ankylosing spondylitis, Grave's disease, Scleroderma, Amyotrophic Lateral Sclerosis, autoimmune hemolytic anemia, psoriasis, vitiligo, eczema, primary biliary cirrhosis, autoimmune prostatitis, Goodpasture's syndrome, autoimmune hepatitis II, celiac disease, ulcerative colitis, thromboembolic syndrome, systemic vasculitides/Wegener's granulomatosis, autoimmune thrombocytopenic purpura, arthritis deformans, Lyme disease arthritis, osteoarthritis, psoriatic arthritis, gout, fibromyalgia, Still's disease, chronic uveitis, chronic back or neck pain and sciatica, Addison's disease, Gaucher's disease, Huntington's disease, muscular dystrophy, cystic fibrosis and idiopathic pulmonary fibrosis. See, e.g., Paul, W. E. (1993) Fundamental Immunology, Third Edition, Raven Press, New York, Chapter 30, pp. 1033-1097; and Cohen, et al. (1994) Autoimmune Disease Models, A Guidebook, Academic Press, 1994.

Accordingly, the invention is further directed to a method for treating chronic inflammation or immune-mediated autoimmunity by administering or delivering to a subject in need of treatment a CAR, a nucleic acid construct or an immune cell of this invention. In particular embodiments, chronic inflammation or immune-mediated autoimmunity is treated by delivering an immune cell. The step of delivering the immune cell to the subject generally involves introducing, e.g., via transduction, transposons or electroporation, a nucleic acid construct of the invention into an isolated immune cell (e.g., an autologous or third party-derived immune cell) and introducing into the subject the transformed/transduced immune cell, thereby effecting inflammatory or immunosuppressive responses in the subject and treating or ameliorating chronic inflammation or immune-mediated autoimmunity.

“Immune cell” as used herein refers to the cells of the mammalian immune system including but not limited to antigen presenting cells, B-cells, basophils, cytotoxic T-cells, dendritic cells, eosinophils, granulocytes, helper T-cells, leukocytes, lymphocytes, macrophages, mast cells, memory cells, monocytes, natural killer cells, neutrophils, phagocytes, plasma cells and T-cells (e.g., naive T cells, central memory T cells, effector memory T cells). In particular embodiments, the immune cell of the invention is a T cell. In certain embodiments, the immune cell of the invention is a CD4+ T cell. In other embodiments, the immune cell is a CD4+ Foxp3+ T cell, i.e., a natural or induced T regulatory cell.

Suitable T cells that can be used include autologous T lymphocyte cells, third party-derived T cells, transformed tumor or xenogenic immunologic effector cells, tumor infiltrating lymphocytes, cytotoxic lymphocytes or other cells that are capable of killing target cells when activated. As is known to one of skill in the art, various methods are readily available for isolating these cells from a subject. For example, using cell surface marker expression or using commercially available kits (e.g., ISOCELL from Pierce, Rockford, Ill.).

It is contemplated that the nucleic acid construct can be introduced into the immune cells as naked DNA or in a suitable vector. Methods of stably transfecting immune cells by electroporation using naked DNA or capped mRNA are known in the art. See, e.g., U.S. Pat. No. 6,410,319. Naked DNA generally refers to the DNA encoding the nucleic acid construct of the invention contained in an expression vector in proper orientation for expression.

Alternatively, a viral vector (e.g., a retroviral vector, adenoviral vector, adeno-associated viral vector, or lentiviral vector) can be used to introduce the nucleic acid construct of the invention into immune cells. Suitable vectors for use in accordance with the method of the invention are non-replicating in the immune cells. A large number of vectors are known that are based on viruses, where the copy number of the virus maintained in the cell is low enough to maintain the viability of the cell. Illustrative vectors include the pFB-neo vectors (STRATAGENE), as well as vectors based on HIV, SV40, EBV, HSV or BPV.

Both lentiviruses and retroviruses have been widely used as gene transfer vectors, and they compose the vector system that is currently used in the majority of clinical gene therapy trials (Sinn, et al. (2005) Gene Ther. 12:1089-1098). However, the lentiviral vectors can be used with both dividing and nondividing cells, result in long-term, stable transgene expression and appear to be less prone to gene silencing (Sinn, et al. (2005) Gene Ther. 12:1089-1098).

Nonviral gene transfer technologies have also been explored for gene therapy. One approach includes the electrotransfer of DNA plasmids using the Sleeping Beauty (SB) transposon/transposase system into primary human immune cells, which has been shown to provide efficient and stable CD19-specific CAR gene expression (Singh, et al. (2008) Cancer Res. 68:2961-71; Maiti, et al. (2013) J. Immunother. 36:112-123). An alternative non-viral approach that does not rely on transgene integration, which uses RNA electroporation, results in transient CAR expression, precluding effective T-cell persistence beyond a week (Zhao, et al. (2006) Mol. Ther. 13:151-159). The use of transient CAR immune cells, which require multiple injections to provide meaningful tumor responses, may reduce the destruction of normal tissues or prevent T cell accumulations to levels that increase the risk of cytokine storms (Zhao, et al. (2010) Cancer Res. 70:9053-61). Moreover, mRNA CAR T cells have been shown to mediate antitumor activity in patients with advanced solid tumors (Beatty, et al. (2014) Cancer Immunology Res. 2:112-20).

Once it is established that the transfected or transduced immune cell is capable of expressing proteins of the nucleic acid construct with the desired regulation and at a desired level, it can be determined whether the one or more CARs are functional in the mammalian cell.

Subsequently, the transduced immune cells are reintroduced or administered to the subject to activate anti-inflammatory or immunosuppresive responses in the subject.

To facilitate administration, the transduced immune cells according to the invention can be made into a pharmaceutical composition or made implant-appropriate for administration in vivo, with appropriate carriers or diluents, which further can be pharmaceutically acceptable. The means of making such a composition or an implant have been described in the art (see, for instance, Remington: The Science and Practice of Pharmacy, Lippincott Williams & Wilkins, 21st edition (2005). Where appropriate, the transduced immune cells can be formulated into a preparation in semisolid or liquid form, such as a capsule, solution, injection, inhalant, or aerosol, in the usual ways for their respective route of administration. Means known in the art can be used to prevent or minimize release and absorption of the composition until it reaches the target tissue or organ, or to ensure timed-release of the composition. Desirably, however, a pharmaceutically acceptable form is employed which does not ineffectuate the cells of the invention. Thus, desirably the transduced immune cells can be made into a pharmaceutical composition containing a balanced salt solution, preferably Hanks' balanced salt solution, or normal saline. Additional examples of carriers or diluents include fats, oils, water, saline solutions, lipids, liposomes, resins, binders, fillers and the like, or combinations thereof. The composition may also include various antioxidants to retard oxidation of one or more component.

A pharmaceutical composition of the invention can be used alone or in combination with other well-established agents useful for inflammation or an autoimmune disease. Whether delivered alone or in combination with other agents, the pharmaceutical composition of the invention can be delivered via various routes and to various sites in a mammalian, particularly human, body to achieve a particular effect. One skilled in the art will recognize that, although more than one route can be used for administration, a particular route can provide a more immediate and more effective reaction than another route. For example, intradermal delivery may be advantageously used over inhalation for the treatment of psoriasis, vitiligo or eczema. Local or systemic delivery can be accomplished by administration comprising application or instillation of the formulation into body cavities, inhalation or insufflation of an aerosol, or by parenteral introduction, comprising intramuscular, intravenous, intraportal, intrahepatic, peritoneal, subcutaneous, or intradermal administration.

Although systemic (intravenous, IV) injection is favored in clinical applications because of its ease of administration several preclinical studies (Carpenito, et al. (2009) Proc. Natl. Acad. Sci. USA 106:3360-3365; Song, et al. (2011) Cancer Res. 71:4617-4627; Parente-Pereira, et al. (2011) J. Clin. Immunol. 31:710-718) suggest that the regional (intratumoral, IT or intraperitoneal, IP) administration of T cells may provide optimal therapeutic effects, which may be in part due to increased T-cell trafficking. For example, it has been shown that CAR T cells remain at the site of inoculation with minimal systemic absorption when delivered via IP or IT routes (Parente-Pereira, et al. (2011) J. Clin. Immunol. 31:710-718). In contrast, after IV administration, CAR immune cells initially reach the lungs and then are redistributed to the spleen, liver, and lymph nodes. In addition, RNA CAR-electroporated immune cells may be particularly suitable for regional administration, due to the transient nature of the CAR expression on the immune cells (Zhao, et al. (2010) Cancer Res. 70:9053-9061). Furthermore, clinical studies have shown the feasibility and safety of both the intratumoral and intraperitoneal injection of immune cells (Canevari, et al. (1995) J. Natl. Cancer Inst. 87:1463-1469; Duval, et al. (2006) Clin. Cancer Res. 12:1229-123680). Overall, a local route of administration of the chimeric immune cells may provide the optimal therapeutic effect and decrease the potential for the “on-target, off-organ” toxicity discussed below.

A composition of the invention can be provided in unit dosage form wherein each dosage unit, e.g., an injection, contains a predetermined amount of the composition, alone or in appropriate combination with other active agents including conventional immunosuppressants and anti-inflammatory agents. The term unit dosage form, as used herein, refers to physically discrete units suitable as unitary dosages for human and animal subjects, each unit containing a predetermined quantity of the composition of the invention, alone or in combination with other active agents, calculated in an amount sufficient to produce the desired effect, in association with a pharmaceutically acceptable diluent, carrier, or vehicle, where appropriate. The specifications for the novel unit dosage forms of the invention depend on the particular pharmacodynamics associated with the pharmaceutical composition in the particular subject.

Desirably an effective amount or sufficient number of the isolated transduced immune cells is present in the composition and introduced into the subject such that long-term, specific, anti-inflammatory or immunosuppressive responses are established to reduce or ameliorate one or more signs or symptoms associated with inflammation or autoimmunity than would otherwise result in the absence of such treatment.

Accordingly, the amount of transduced immune cells administered should take into account the route of administration and should be such that a sufficient number of the transduced immune cells will be introduced so as to achieve the desired therapeutic response. Furthermore, the amounts of each active agent included in the compositions described herein (e.g., the amount per each cell to be contacted or the amount per certain body weight) can vary in different applications. In general, the concentration of transduced immune cells desirably should be sufficient to provide in the subject being treated at least from about 1×10⁶ to about 1×10⁹ transduced immune cells, even more desirably, from about 1×10⁷ to about 5×10⁸ transduced immune cells, although any suitable amount can be utilized either above, e.g., greater than 5×10⁸ cells, or below, e.g., less than 1×10⁷ cells. The dosing schedule can be based on well-established cell-based therapies (see, e.g., Topalian & Rosenberg (1987) Acta Haematol. 78 Suppl 1:75-6; U.S. Pat. No. 4,690,915) or an alternate continuous infusion strategy can be employed.

Any of the compositions described herein may be included in a kit. The kits will thus include, in suitable container means, cells, proteins or nucleic acid constructs or related reagents of the present invention. In some embodiments, the kit further includes an additional immunosuppressive or anti-inflammatory agent. Examples of available immunosuppressants and anti-inflammatory agents include, but are not limited to, cyclosporine A, cyclophosphamide, prednisone, tacrolimus (FK506), nonsteroidal anti-inflammatory agents such as aspirin, ibuprofen, diclofenac, etodolac, fenoprofen, flurbiprofen, naproxen, and oxaprozin. In certain embodiments, the additional agent may be combined with the nucleic acid construct(s) or immune cells of the invention or may be provided separately in the kit. In some embodiments, means of taking a sample from an individual and/or of assaying the sample may be provided in the kit. In certain embodiments the kit includes cells, buffers, cell media, vectors, primers, restriction enzymes, salts, and so forth, for example.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. Such containers may include injection or blow-molded plastic containers into which the desired vials are retained.

When the components of the kit are provided in one and/or more liquid solutions, the liquid solution is an aqueous solution, with a sterile aqueous solution being particularly preferred. The compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit. However, the components of the kit may be provided as dried powder(s). When reagents and/or components are provided as a dry powder, the powder can be reconstituted by the addition of a suitable solvent. It is envisioned that the solvent may also be provided in another container means.

The following non-limiting examples are provided to further illustrate the present invention.

### Example 1: CAR Construct with CTLA-4 Signaling Domain

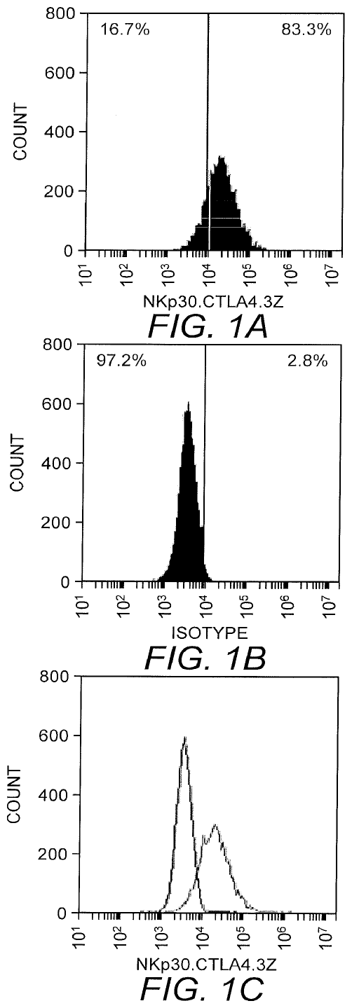

A CAR construct composed of nucleic acids encoding the NKp30 antigen-specific targeting domain fused to the transmembrane and cytoplasmic domains of CTLA-4 and cytoplasmic domain of CD3ζ was prepared. This construct was highly expressed on packaging cells line E86 (FIGS. 1A-1C). It is expected that this CAR construct, when transuded into CD4+ CD25+ CD127− Foxp3+ Treg cells, wild readily express the CAR on the cell surface.

1. A chimeric antigen receptor comprising at least one signaling domain of CD44.

2. The chimeric antigen receptor of claim 1, wherein the chimeric antigen receptor comprises an antigen targeting domain or recognition domain that binds an antigen or ligand at a site of inflammation or autoimmunity.

3. The chimeric antigen receptor of claim 1, wherein the chimeric antigen receptor comprises a single chain variable fragment that binds an antigen or ligand at a site of inflammation or autoimmunity.

4. The chimeric antigen receptor of claim 1, further comprising at least one signaling domain of a co-inhibitory receptor selected from Cytotoxic T-Lymphocyte Antigen 4 (CTLA-4), Lymphocyte-Activation Gene 3 (LAG-3), Programmed cell death protein 1 (PD-1), T cell Immunoglobulin Mucin-3 (TIM-3), T-cell immunoreceptor with immunoglobulin (TIGIT), Band T-lymphocyte Attenuator (BTLA), leukocyte immunoglobulin-like receptor subfamily B member 4 (LILRB4), LILRB3, CD160, 2B4, Leukocyte-Associated Immunoglobulin-like Receptor 1 (LAIR-1), CD66a, and neuropilin-1 (NRp1).

5. A nucleic acid construct comprising nucleic acids encoding the chimeric antigen receptor of claim 1.

6. The nucleic acid construct of claim 5, wherein the construct comprises a vector.

7. An immune cell comprising nucleic acids encoding the chimeric antigen receptor of claim 1.

8. The immune cell of claim 7, wherein the cell is a T lymphocyte.

9. A method for treating chronic inflammation or immune-mediated autoimmunity comprising delivering to a subject in need of treatment an immune cell of claim 7 thereby treating the subject's chronic inflammation or immune-mediated autoimmunity.

10. A kit comprising the nucleic acid construct of claim 5.

11. A kit comprising one or more immune cells of claim 7.

12. The immune cell of claim 8, wherein the T lymphocyte is a Treg cell.
